(19) United States
(12) Patent Application Publication
(10) Pub. No.: US 2014/0294753 A1
(43) Pub. Date: Oct. 2, 2014

(54) NOVEL USE OF GANODERMIC ACIDS FOR TREATING CANCER

(71) Applicant: DOUBLE CRANE BIOTECHNOLOGY CO. LTD, New Taipei City (TW)

(72) Inventors: Shwu-Bin LIN, Taipei City (TW); Cheng-Po HUANG, Hsinchu City (TW); Teng-Hai CHEN, Tainan City (US)

(73) Assignee: Double Crane Biotechnology Co. LTD, New Taipei City (TW)

(21) Appl. No.: 13/854,024

(22) Filed: Mar. 29, 2013

(51) Int. Cl. A61K 31/575; A61K 45/06

(52) U.S. Cl. CPC A61K 31/575; A61K 45/06
USPC 424/85.1; 514/179; 514/171

(57) ABSTRACT

Novel Uses of a small molecule, ganodermic acid S (GMAS), are disclosed herein. The GMAS is useful as a lead compound for manufacturing a medicament or a pharmaceutical composition for treating cancer, including metastatic or drug-resistant cancer, in a patient.

### BACKGROUND OF THE INVENTION

1. Field of the Invention

The present disclosure relates to novel use of ganodermic acid S (GMAS) as a lead compound for manufacturing a medicament or a pharmaceutical composition for treating cancer.

2. Description of Related Art

Edible fungus has long been used as a nutritional aid or health food in the Southeastern Asia, with Ling-Zhi, the Chinese name for one form of mushroom Ganoderma lucidum, being the most popular and oldest mushroom known to have medicinal usages for thousands years. Various active compounds have been isolated from Ganoderma including triterpenoids, polysaccharides, proteins, nucleic acids, polypeptides and phyto-sterols and etc. Among them, triterpenoids are the most important components in Ling-Zhi with significant pharmacological activities such as inhibition of cholesterol synthesis, antitumor, antihypertensive and etc. Triterpenoids are generally known to include various types of ganoderic acids (GAs), ganodermic acids (GMAS), ganoderic alcohols, ganoderic ketones and ganoderic aldehydes and etc. Prior studies have demonstrated that GAs possess cytotoxic and/or anti-proliferative effects against tumor cells. For example, ganoderic acid D (GAD) was found to inhibit the proliferation of HeLa human cervical carcinoma (Yue et al., Mol Cell Proteomics (2008) 7, 949-961); ganoderic acids A and H (GAA and GAH) were demonstrated to suppress growth and invasive behavior of breast cancer cells (Jiang et al., Int J Mol Med (2008) 21, 577-584); ganoderic acid X (GAX) was found to inhibit topoisomerases and induced apoptosis in liver cancer cells (Li et al., Life Sci. (2005) 77, 252-265); and ganoderic acid Me (GAMe) effectively inhibited tumor growth, and lung metastasis (Wang et al., Int Immunopharmacol (2007) 7, 864-870). However, little attention has been paid to the bioactivity of GMAS, particularly, ganodermic acid S (GMAS), except that GMAS was found to induce aggregation of platelets (Wang et al., Biochim. Biophys. Acta. (1989) 986, 151-160), inhibit function of platelets (Wang et al., Biochem. J. (1991) 277 (Pt 1), 189-197), as well as the signaling cellular responses induced by thromboxane A2 (Su et al., Biochem. Pharmacol. (1999) 58, 587-595; Su et al., Biochim. Biophys. Acta. (1999b) 1437, 223-234) or prostaglandin E1 (Su et al., Thromb. Res. (1999c) 99, 135-145) in platelets.

Inventors of this application unexpected identify that GMAS also exhibits anti-proliferative effects toward certain tumors, including drug resistant cancers, hence may be used as a lead compound in the treatment or prophylaxis of cancers.

### SUMMARY

The present disclosure is based, at least in part, unexpected discovery that ganodermic acid S (GMAS) isolated from the fruit bodies of Ganoderma lucidum may retard the growth or metastasis of cancerous cells. The results of this invention suggest that GMAS is a potential lead compound for use as a therapeutic agent for treatment or prophylaxis of cancers, including cancers that are drug-resistant.

Accordingly, it is the first aspect of this disclosure to provide a method of treating cancer in a subject. The method comprises administering to the subject a therapeutically effective amount of GMAS or a pharmaceutically acceptable salt thereof. The cancer suitable for treating by the method of this disclosure is selected from the group consisting of colon cancer, hepatic cancer, breast cancer, or lung cancer. In one preferred example, the lung cancer is resistant to gefitinib. In another example, the lung cancer is metastatic. In still another example, the colon cancer is metastatic. The subject may be a mammal, preferably a human.

In some embodiments, the method further comprises subjecting the subject to radiation treatment after administering the compound of this invention to the subject. In one example, the lung cancer is resistant to gefitinib. In another example, the lung cancer is metastatic. In still another example, the colon cancer is metastatic.

It is therefore the second aspect of this disclosure to provide a use of GMAS for manufacturing a medicament or a pharmaceutical composition for treating cancer; the medicament or the pharmaceutical composition comprises a therapeutically effective amount of GMAS or a pharmaceutically acceptable salt thereof; and a pharmaceutically acceptable excipient.

The compound of this invention, specifically GMAS, is present at a level of about 0.1% to 99% by weight, based on the total weight of the pharmaceutical composition. In some embodiments, the compound of this invention is present at a level of at least 1% by weight, based on the total weight of the pharmaceutical composition. In certain embodiments, the compound of this invention is present at a level of at least 5% by weight, based on the total weight of the pharmaceutical composition. In still other embodiments, the compound of this invention is present at a level of at least 10% by weight, based on the total weight of the pharmaceutical composition. In still yet other embodiments, the compound of this invention is present at a level of at least 25% by weight, based on the total weight of the pharmaceutical composition.

In some embodiments, the medicament or the pharmaceutical composition of this invention further includes an agent that is known to improve the treatment of cancer. Examples of such agent include, but are not limited to, anti-cancer drug, angiogenesis inhibitor, anti-virus agent, analgesic, anti-anemia drug, cytokine, granulocyte colony-stimulating factor (G-CSF), anti-nausea drug, and the like.

The details of one or more embodiments of the invention are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The present description will be better understood from the following detailed description read in light of the accompanying drawings, wherein:

FIG. 1 illustrates the results of varying doses of GMAS on the cell activity of (A) human lung cancer cell line A549, (B) human colon cancer cell line HCT-116, (C) human hepatoma cell line Huh7, and (D) human breast cancer cell line MDA-MB-231, in accordance with one embodiment of this invention;

FIG. 2A illustrates the results of varying doses of gefitinib on cell activity of human non-small cell lung cancer cell PC-9 and gefitinib-resistant human non-small cell lung cancer cell line PC-9 (PC9-IR) in accordance with one embodiment of this invention;

FIG. 2B illustrates the results of varying doses of GMAS on cell activity of human non-small cell lung cancer cell PC-9 and gefitinib-resistant human non-small cell lung cancer cell line PC-9 (PC9-IR) in accordance with one embodiment of this invention;

FIG. 3 illustrates the results of a wound healing assay measuring the effect of 25 or 50 μM GMAS on the healing of wounds in a culture of HCT-116 cells in accordance with one embodiment of this invention, in which (A) are photographs taken at various time points during a wound healing assay, and (B) is a bar graph of th relative migrating area measured in the wound healing assay; and

FIG. 4 illustrates the results of a wound healing assay measuring the effect of 5 or 10 GMAS on the healing of wounds in a culture of PC-9 cells in accordance with one embodiment of this invention, in which (A) are photographs taken at various time points during a wound healing assay, and (B) is a bar graph of th relative migrating area measured in the wound healing assay.

### DETAILED DESCRIPTION OF PREFERRED EMBODIMENTS

The detailed description provided below in connection with the appended drawings is intended as a description of the present invention and is not intended to represent the only forms in which the present invention may be constructed or utilized.

In the context of this disclosure, a number of terms shall be used.

The terms “treatment” and “treating” are used herein to include preventative (e.g., prophylactic), curative, or palliative treatment that results in a desired pharmaceutical and/or physiological effect. Preferably, the effect is therapeutic in terms of partially or completely curing or preventing the growth of tumor cells. Also, the term “treating” as used herein refers to application or administration of the compound of the present disclosure to a subject, who has a medical condition, a symptom of the condition, a disease or disorder secondary to the condition, or a predisposition toward the condition, with the purpose to partially or completely alleviate, ameliorate, relieve, delay onset of, inhibit progression of, reduce severity of, and/or reduce incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. As used herein, the symptom, disease, disorder or condition may be solid tumor or metastatic tumor. Treatment is generally “effective” if one or more symptoms or clinical markers are reduced as that term is defined herein.

The term “prophylaxis” as used herein means prevention against a future event. In the context of prophylaxis against tumor cells or tumor cell metastasis that may potentially occur as a consequence of a surgical or diagnostic procedure, the prophylactic administration can occur before, contemporaneous with, and/or after the procedure.

The term “an effective amount” as used herein refers to an amount effective, at dosages, and for periods of time necessary, to achieve the desired therapeutically desired result with respect to the treatment of cancer.

The terms “compounds”, “compositions”, “agent” or “medicament” are used interchangeably herein to refer to a compound or a composition of which, when administered to a subject such as a human or an animal induces a desired pharmacological and/or physiological effect by local and/or systemic action.

The term “administered”, “administering” or “administration” are used interchangeably herein to refer means either directly administering a compound or a composition of the present invention, or administering a prodrug, derivative or analog which will form an equivalent amount of the active compound within the body.

The term “subject” or “patient” refers to an animal including the human species that is treatable with the compositions and/or methods of the present invention. The term “subject” or “patient” intended to refer to both the male and female gender unless one gender is specifically indicated. Accordingly, the term “subject” or “patient” comprises any mammal, preferably a human, which may benefit from treatment by the compound of this disclosure.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The present disclosure is based, at least in part, unexpected discovery that ganodermic acid S (GMAS) possesses anti-proliferative and/or anti-metastatic activities toward cancerous cells, including drug-resistant cancerous cells. Therefore, GMAS is a potential lead compound for use as the therapeutic agent for the treatment or prophylaxis of cancers.

Shown below is the chemical structure of GMAS of this disclosure.

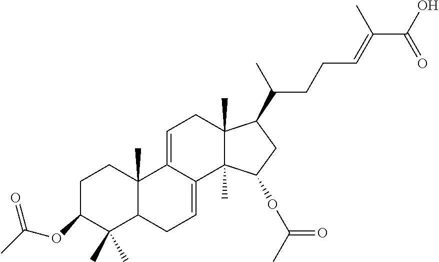

GMAS of this invention is produced by Ganoderma spp. and can be purified from the fruit bodies of Ganoderma lucidum by methods well known in the art, for example, the method described by Hirotani et al (Phytochemistry (1987), 26(10), 2797-2803). Alternatively, GMAS may also be isolated from the mycelium taken from the cultivating bags with solid nutrients for cultivating Ganoderma lucidum in according to the method described in Taiwan Patent No. 1381844, issued to Chen et al on Jan. 11, 2013. Whether the raw material used for isolating GMAS is the fruit bodies or the mycelia, such method in general involves extracting the plant with a solvent, preferably an alcoholic solution, at a temperature above room temperature; followed by subjecting the extract with column chromatography, which includes but is not limited to, high performance liquid chromatography (HPLC), reverse phase liquid chromatography and etc.; and concentrating and drying, until a dried powder is obtained.

Accordingly, this disclosure provides a method of treating cancer in a subject. The method includes administering to the subject an effective amount of the compound described above or a pharmaceutically acceptable salt thereof. The compound of this disclosure is effective in treating cancer by suppressing the growth of cancerous cells and/or preventing them from metastatic. Cancer that may be treated by the compound of this disclosure includes colon cancer, hepatic cancer, breast cancer, or lung cancer. Preferably, the compound of this disclosure is employed to treat drug-resistant cancers, such as gefitinib resistant cancers.

As used herein, drug-resistance refers to a state of cancer in which, having developed resistance to a single drug. For example, a cancer that has developed drug-resistance can show resistance to vinca alkaloids (e.g., vinblastine, vincristine, and vinorelvine); anthracyclines (e.g., doxorubicin, daunorubicin, and idarubicin); microtubule-stabilizing drug paclitaxel; drugs that target tyrosine kinases (TKs) activity (e.g., dasatinib, nilotinib, matinib, and gefitinib).

In one preferred example, the cancer that may be treated by the compound of this disclosure is lung cancer, which has developed drug-resistance to another FDA approved drug, gefitinib. In other example, the cancer is colon cancer, particularly metastatic colon cancer. In still another example, the cancer is lung cancer, particularly metastatic lung cancer.

In some embodiments, the effective amount of the compounds of this invention administered to the subject is from about 1 to 100 mg/Kg body weight of the subject by oral ingestion, intravenous or intramuscular injection. The amount is administered to the subject at about 10, 20, 30, 40, 50, 60, 70, 80, 90 or 100 mg/Kg body weight of the subject per day, preferably about 30 to 70 mg/Kg body weight of the subject, such as 30, 40, 50, 60 or 70 mg/Kg body weight of the subject per day. The dose can be administered in a single dosage, or alternatively in more than one dosage.

In some embodiments, the method further includes the step of subjecting the cancer to a radiation treatment after administering the compounds of the invention.

In some embodiments, the method further includes the step of administering another agent that is known to improve the treatment of cancer, before, together with and/or after administering the compound of this invention. Examples of such agent include, but are not limited to, anti-cancer drug, anti-angiogenesis agent, anti-virus agent, analgesic, anti-anemia drug, cytokine, granulocyte colony-stimulating factor (G-CSF), and anti-nausea drug and the like.

Examples of anti-cancer drug include, but are not limited to, paclitaxel, docetaxel, camptothecin (CPT), topotecan (TPT), irinotecan (CPT-11), Doxorubicin, daunorubicin, epirubicin, fluorouracil, cis-platin, cyclophosphamide, vinblastine, vincristine, ifosfamide, melphalan, mitomycin, methotrexate, mitoxantrone, teniposide, etoposide, bleomycin, leucovorin, cytarabine, dactinomycin, streptozocin, combretastatin A4-phosphate, SU5416, and the like. Examples of anti-angiogenesis agent include, but are not limited to, DS 4152, TNP-470, SU6668, endostatin, 2-methoxyestradiol, angiostatin, thalidomide, tetrathiomolybdate, linomide, IL-12, and the like. Examples of anti-virus agent include, but are not limited to, amantadine, rimantadine, and the like. Examples of analgesic include, but are not limited to, paracetamol such as para-acetylaminophenol, non-steroidal anti-inflammatory drug (NSAID) such as salicylates, and opioid drugs such as morphine and opium. Example of anti-anemia drug includes, and is not limited to, erythropoietin.

This disclosure also provides a pharmaceutical composition for treating cancer; the composition comprises a therapeutically effective amount of a compound of this disclosure as shown above; and a pharmaceutically acceptable excipient.

Generally, the compound of this invention is present at a level of about 0.1% to 99% by weight, based on the total weight of the pharmaceutical composition. In some embodiments, the compound of this invention is present at a level of at least 1% by weight, based on the total weight of the pharmaceutical composition. In certain embodiments, the compound of this invention is present at a level of at least 5% by weight, based on the total weight of the pharmaceutical composition. In still other embodiments, the compound of this invention is present at a level of at least 10% by weight, based on the total weight of the pharmaceutical composition. In still yet other embodiments, the compound of this invention is present at a level of at least 25% by weight, based on the total weight of the pharmaceutical composition.

In some embodiments, the medicament of said pharmaceutical composition of this invention further includes an agent that is known to improve the treatment of cancer. Examples of such agent include, and are not limited to, anti-cancer drug, anti-angiogenesis agent, anti-virus agent, analgesic, anti-anemia drug, cytokine, granulocyte colony-stimulating factor, anti-nausea drug and the like.

The medicament or said pharmaceutical composition is prepared in accordance with acceptable pharmaceutical procedures, such as described in Remington's Pharmaceutical Sciences, 17^{th} edition, ed. Alfonoso R. Gennaro, Mack Publishing Company, Easton, Pa. (1985). Pharmaceutically acceptable excipients are those that are compatible with other ingredients in the formulation and biologically acceptable.

The compounds of this invention (e.g., GMAS) may be administered by any suitable route, for example, orally in capsules, suspensions or tablets or by parenterally administration. Parenterally administration can include, for example, systemic administration such as intramuscular, intravenous, subcutaneous, or intraperitoneal injection. The compound can also be administered transdermally either topically or by inhalation (e.g., intrabronichial, intranasal, oral inhalation or intranasal drops), or rectally, alone or in combination with conventional pharmaceutically acceptable excipients. In preferred embodiments, the compounds of this invention are administered orally (e.g., dietary) to the subject.

For oral administration, the compounds of the present invention may be formulated into tablets containing various excipients such as microcrystalline cellulose, sodium citrate, calcium carbonate, dicalcium phosphate, and glycine; along with various disintegrants such as starch, alginic acid and certain silicates; together with granulation binders like polyvinylpyrrolidone, sucrose, gelatin and acacia. Additionally, lubricating agents such as magnesium stearate, sodium lauryl sulfate and talc may be added. Solid composition may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and if so desired, emulsifying and/or suspending agents as well, together with diluents such as water, ethanol, propylene glycol, glycerin and a combination thereof.

For parenteral administration, the compounds of the present invention may be formulated into liquid pharmaceutical compositions, which are sterile solutions, or suspensions that can be administered by, for example, intravenous, intramuscular, subcutaneous, or intraperitoneal injection. Suitable diluents or solvent for manufacturing sterile injectable solution or suspension include, but are not limited to, 1,3-butanediol, mannitol, water, Ringer's solution, and isotonic sodium chloride solution. Fatty acids, such as oleic acid and its glyceride derivatives are also useful for preparing injectables, as are natural pharmaceutically-acceptable oils, such as olive oil or castor oil. These oil solutions or suspensions may also contain alcohol diluent or carboxymethyl cellulose or similar dispersing agents. Other commonly used surfactants such as Tweens or Spans or other similar emulsifying agents or bioavailability enhancers that are commonly used in manufacturing pharmaceutically acceptable dosage forms can also be used for the purpose of formulation.

For topical administration, the medicament or said pharmaceutical compositions of this invention may be formulated into a variety of dosage forms for topical application. A wide variety of dermatologically acceptable inert excipients well known to the art may be employed. The topical compositions may include liquids, creams, lotions, ointments, gels, sprays, aerosols, skin patches, and the like. Typical inert excipients may be, for example, water, ethyl alcohol, polyvinyl pyrrolidone, propylene glycol, mineral oil, stearyl alcohol and gel-producing substances. All of the above dosages forms and excipients are well known to the pharmaceutical art. The choice of the dosage form is not critical to the efficacy of the composition described herein.

For transmucosal administration, the medicament or said pharmaceutical compositions of this invention may also be formulated in a variety of dosage forms for mucosal application, such as buccal and/or sublingual drug dosage units for drug delivery through oral mucosal membranes. A wide variety of biodegradable polymeric excipients may be used that are pharmaceutically acceptable, provide both a suitable degree of adhesion and the desired drug release profile, and are compatible with the active agents to be administered and any other components that may be present in the buccal and/or sublingual drug dosage units. Generally, the polymeric excipient comprises hydrophilic polymers that adhere to the wet surface of the oral mucosa. Examples of polymeric excipients include, but are not limited to, acrylic acid polymers and copolymers; hydrolyzed polyvinylalcohol; polyethylene oxides; polyacrylates; vinyl polymers and copolymers; polyvinylpyrrolidone; dextran; guar gum; pectins; starches; and cellulosic polymers.

Accordingly, this invention also provides methods of treating mammals, preferably humans, for cancer, which comprises the administration of the medicament or said pharmaceutical composition of this invention that contains a compound of this invention. Such medicament or composition is administered to a mammal, preferably human, by any route that may effectively transports the active ingredient(s) of the composition to the appropriate or desired site of action, such as oral, nasal, pulmonary, transdermal, such as passive or iontophoretic delivery, or parenteral, e.g., rectal, depot, subcutaneous, intravenous, intramuscular, intranasal, ophthalmic solution or an ointment. Further, the administration of the compound of this invention with other active ingredients may be concurrent or simultaneous.

It will be appreciated that the dosage of compounds of the present invention will vary from patient to patient not only for the particular compound or composition selected, the route of administration, and the ability of the compound (alone or in combination with one or more drugs) to elicit a desired response in the patient, but also factors such as disease state or severity of the condition to be alleviated, age, sex, weight of the patient, the state of being of the patient, and the severity of the pathological condition being treated, concurrent medication or special diets then being followed by the patient, and other factors which those skilled in the art will recognize, with the appropriate dosage ultimately being at the discretion of the attendant physician. Dosage regimens may be adjusted to provide the improved therapeutic response. A therapeutically effective amount is also one in which any toxic or detrimental effects of the compound or composition are outweighed by the therapeutically beneficial effects. Preferably, the compounds or compositions of the present invention are administered at a dosage and for a time such that the number and/or severity of the symptoms are decreased.

The present invention will now be described more specifically with reference to the following embodiments, which are provided for the purpose of demonstration rather than limitation.

### EXAMPLES

### Materials and Methods

### Cell and Culture

Cell lines used in the present disclosure include human lung adenocarcinoma cell line A549, human hepatocarcinoma cell line Huh7, human colon carcinoma cell line HCT116, human non-small cell lung cancer cell line PC-9, gefitinib-resistant human non-small cell lung cancer cell line PC-9 (PC9-IR), and human breast carcinoma cell line MDA-MB-231.

Each cell lines were cultured and maintained in Dulbecco's modified Eagle media (DMEM) supplemented with 1% fetal bovine serum (FBS), 100 IU/ml penicillin, 100 ng/ml streptomycin, 2 mM glutamine, non-essential amino acids and sodium pyruvate in 5% CO₂ at 37° C. Cells were grown and maintained in Petri dishes (each was 10 cm in diameter) until reached 80% confluence, then were subject to cell passages. Briefly, cells were first washed with phosphate buffer solution (PBS, 3 ml) once, then treated with 0.05% Trypsin/0.025% EDTA solution (1 mL) for 5 min so that the attached cells become suspended. Collected the suspended cells, added 2 mL fresh culture media to neutralize any remaining activity of trypsin. Adjusted the cell density by adding appropriate amounts of culture medium to the cell suspension, which was then used to seed the culture plates. The plates were then returned to the incubator and cultured in accordance with the steps described above.

### Cell Activity Analysis

Cells were seeded in 96-well plates with a density of 3,000 cells/well and cultured in accordance with the procedures described above. On the day when cell activity analysis was to be conducted, cells were first treated with various concentrations of GMAS, or gefitinib for at least 48 hrs, before subjecting them to acid phosphatase (ACP) analysis.

Acid Phosphatase analysis

Cell activity may be derived from the acid phosphatase (ACP) activity of a cell. Live cells possess aboundant amounts of ACP, which converts its substrate, p-nitrophenyl phosphate (p-NPP) to p-nitrophenol (p-NP) with a maximum adsorption occurs at the wavelength of 405 nm; hence, ACP activity may be used as an indication of cell activity.

In operation, cultured cells in each well were washed with PBS (200 μl), then a reacting solution (100 μl) (10 mM p-NPP, 0.1 M sodium acetate, 0.1% Triton X-100, pH 5.5) was added and allowed to react with the cells for 30-40 min at 37° C. The reaction was subsequently stopped with a basic solution (10 μl, 0.1N NaOH). Adsorption at the wavelength of 405 nm was then measured, and the concentration that inhibited 50% cell activity (IC₅₀) was derived from the absorbance measurement.

### Wound Healing Assay

Wound healing assay is one of the earliest developed methods to study directional cell migration in vitro, by mimicking cell migration during wound healing in vivo. The basic steps involve creating a “wound” in a cell monolayer, capturing the images at the beginning and at regular intervals during cell migration to close the wound, and comparing the images to quantify the migration rate of the cells.

Briefly, the assay was carried out using IBIDI culture-inserts and 24-well plates (IBIDI GmbH, Martinsried, Germany). The culture-inserts were respectively placed into each well. Aliquots of 704 medium containing 3×10⁴ cells (PC-9, MDA-MB-231 or CL1-5 cells) were added into the two reservoirs of the same insert and incubated for 8 hrs, then the inserts were gently removed from each wells, then culture medium (500 μL, 10% FBS) containing GMAS (25 or 50 μM) or solvent vehicle (i.e., the control) was added to each well. The cells in the culture wells were photographed respectively at 0, 12, 24, 48 and 72 hrs using an inverted microscope. Finally, the migration area or the “wound” area was quantified by Metamorph software (Molecular Devices).

### Example 1

### Effects of GMAS on the Activity of Cancer Cells

### 1.1 GMAS Inhibits Various Types of Cancer Cells

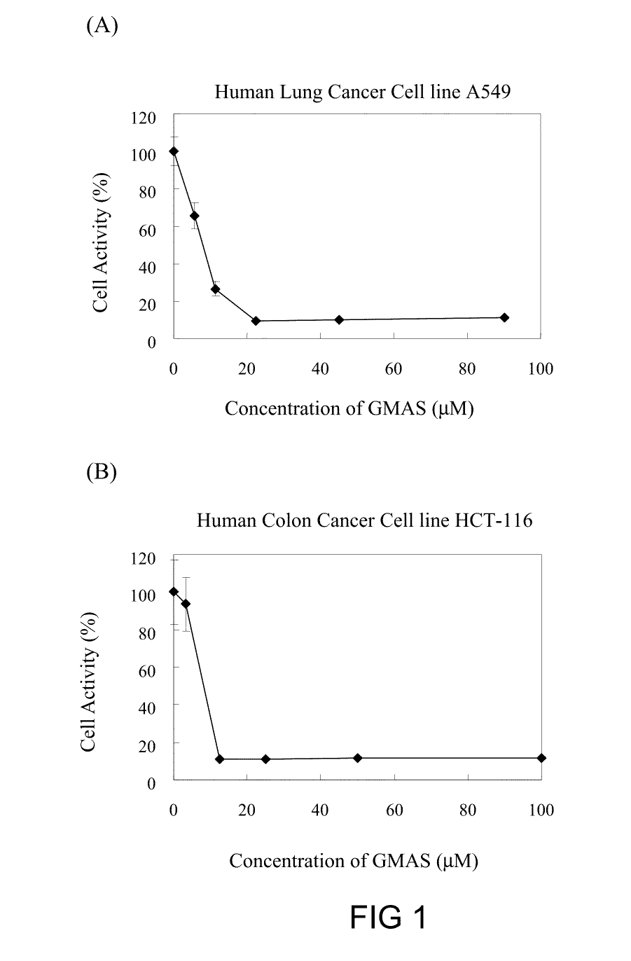

The effects of GMAS on various cancer cell lines, including colon cancer, hepatoma, breast cancer, and lung cancer, were respectively assessed by cell activity analysis using ACP activity as an indicator. Results are depicted in FIG. 1.

As illustrated in FIG. 1, GMAS dose-dependently inhbited cell activity in four cancer cells that were tested, including lung cancer cell line A549, colon cancer cell line HCT-116, hepatoma cell line Huh7 and breast cancer cell line MDA-MB-231. The respective doses in which 50% cell activity inhibited by GMAS (IC₅₀) are summarized in Table 1.

TABLE 1

| | Cell Line | | | |
|---|---|---|---|---|
| | Colon Cancer | Hepatoma | Breast Cancer | Lung Cancer |
| | HCT-116 | Huh-7 | MDA-MB-231 | A549 |
| IC₅₀ (μM) | 8.0 | 19.1 | 20.4 | 7.9 |

### 1.2 GMAS Inhibits the gefitinib-Resistant Cancer Cells

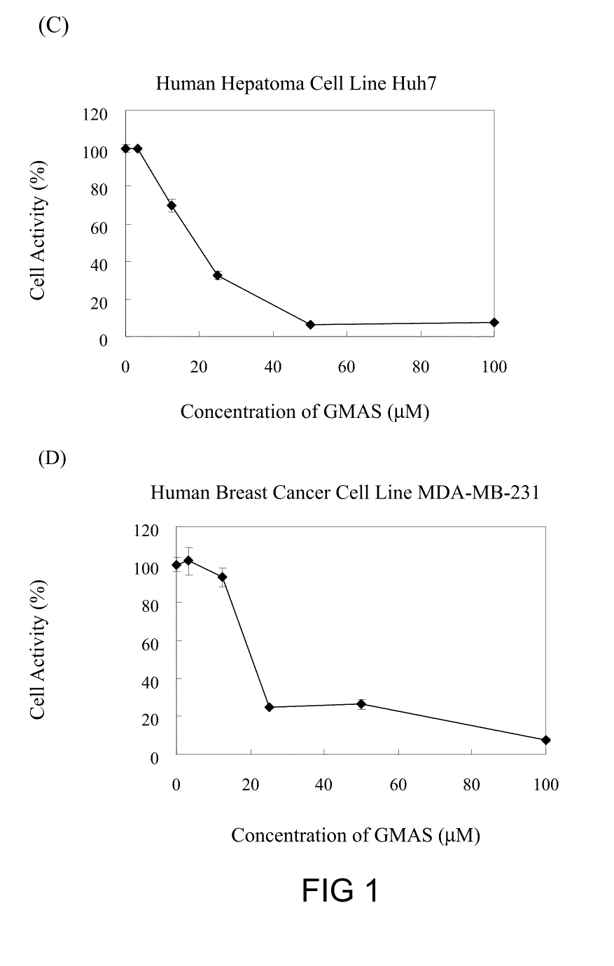

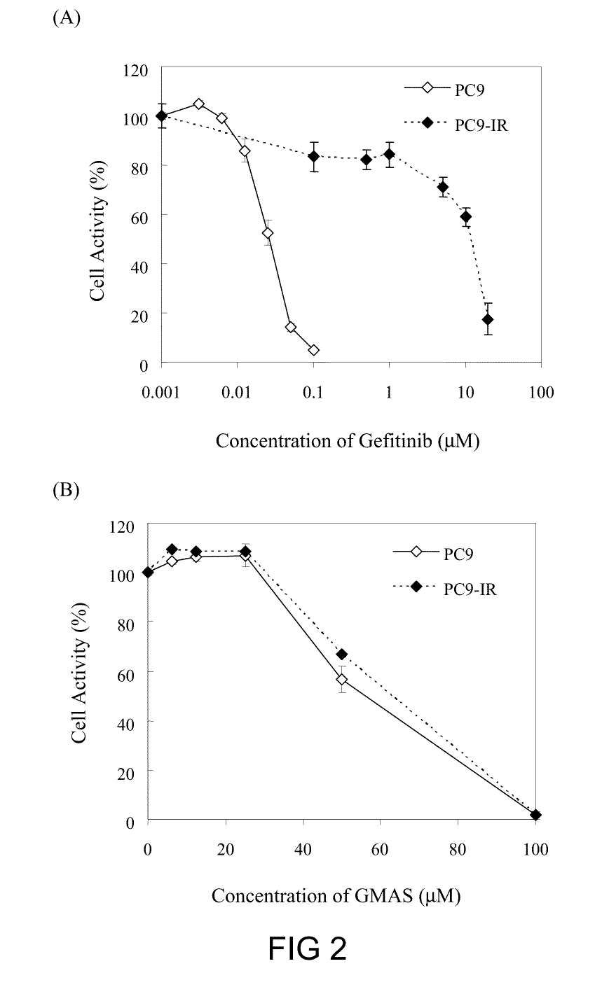

The effects of GMAS on gefitinib-resistant cancer cell line PC9-IR (PC9-IR) were also investigated in accordance with similar procedures described in example 1.1. Results are illustrated in FIG. 2, and the doses in which 50% cell activity inhibited by GMAS or gefitinib (IC₅₀), as well as resistant ratio (RR), which determines the susceptibiity of the two cell lines to the tested chemicals, are summarized in Table 2. RR is an expression of the relative susceptiblity of one cell line towards a test compound, by dividing IC₅₀ of one cell line over that of the other.

TABLE 2

IC₅₀ (μM)

| Cell Line | Gefitinib | GMAS |
|---|---|---|
| PC-9 | 0.0267 | 56.1 |
| PC9-IR | 12.6 | 62.8 |
| Resistant Ratio | 456.0 | 1.1 |
| (PC9-IR/PC9) | | |

Respective cell activities of PC-9 and PC9-IR cell lines were suppressed by gefitinib after being treated with gefitinib for 72 hrs, with IC₅₀ respectively at 0.0267 μM and 12.6 μM. GMAS were also effective in suppressing the cell activity of both cell lines, with similar IC₅₀ at around 60 μM (56.1 vs 62.8 μM). However, when compared with resistant ratio, it is clear that PC-9 cell is more susceptible to gefitinib than the resistant line, PC9-IR; by contrast, the effect of GMAS appears to be the same to both cell lines, in other words, GMAS is as effective in killing drug-resistant cells as in the none drug-resistant cancer cells.

### Example 2

### GMAS Inhibits the Migration of Cancer Cells

### 2.1 GMAS Inhibits the Migration of Colon Cancer Cells

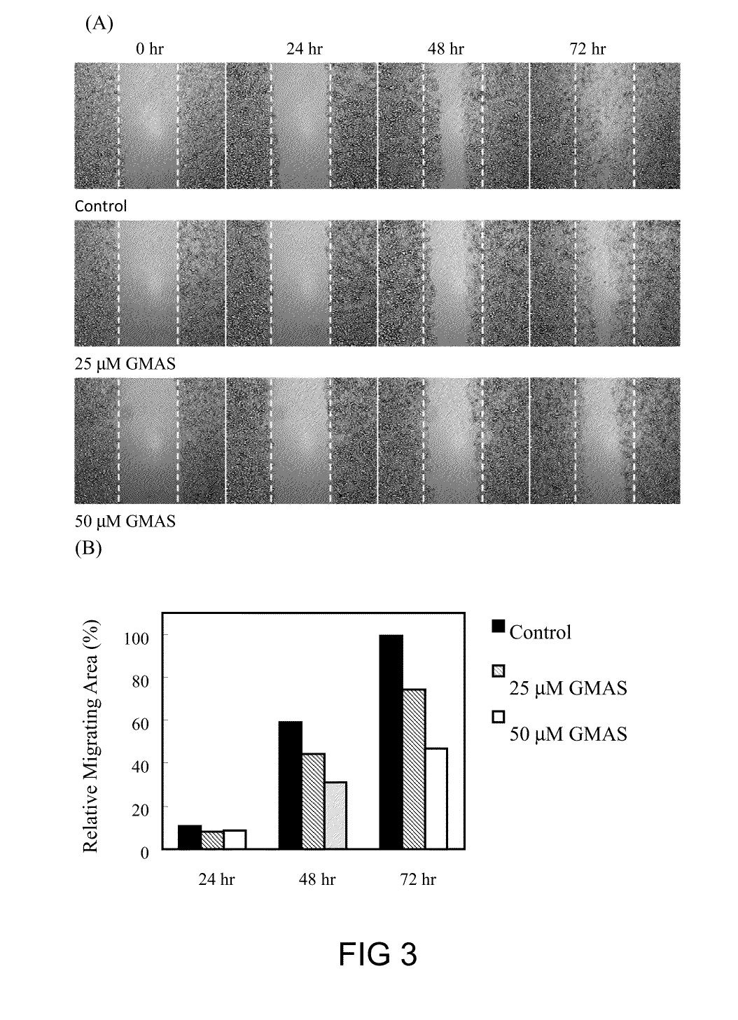

The effect of GMAS on the migration capability of colon cancer cells was assessed using the wound healing assay as described in the section of “Materials and Methods”. Results are illustrated in FIG. 3.

As depicted in FIG. 3, a wound was created in the colon cancer cell monolayer, and migration of colon cancer cells started to take effect after being left in the culture for 48 hrs, with significant “wound” closing effect being observed after 72 hrs. The wound closing phenomenon was slightly inhibited when treated with 25 μM GMAS, and significantly suppressed when treated with 50 μM GMAS.

### 2.2 GMAS Inhibits the Migration of Lung Cancer Cells

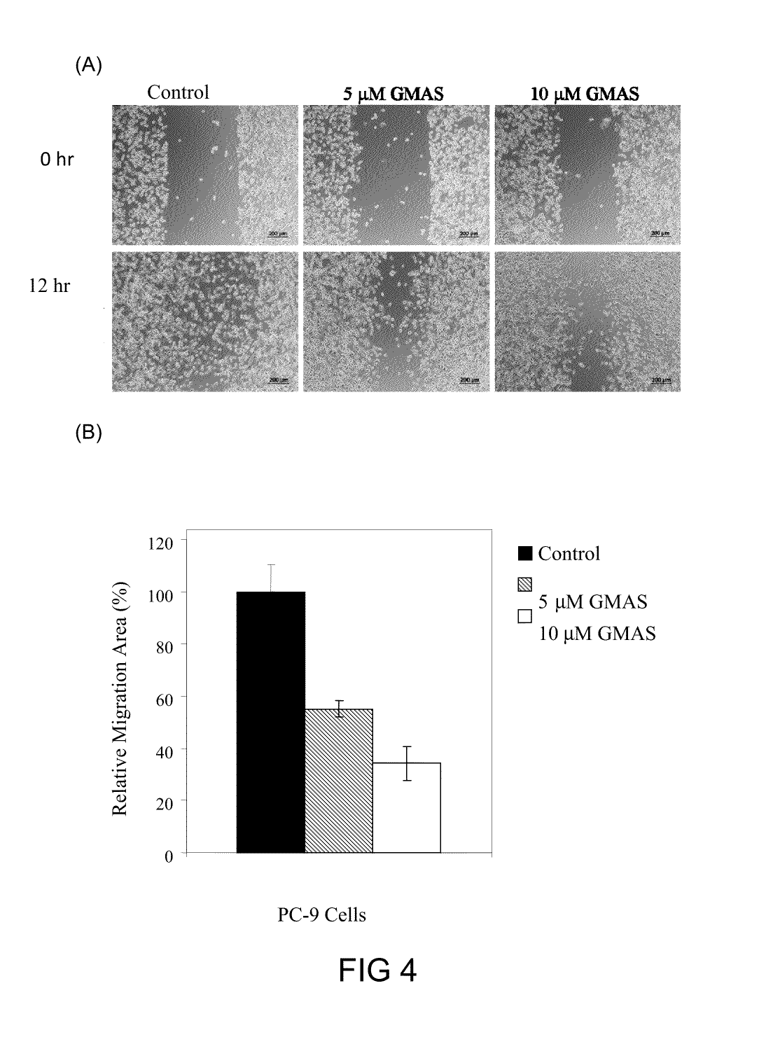

GMAS exhibited similar inhibitory effects on the migration capability of lung cancer cells, with 5 μM GMAS being sufficient enough to reduce 50% of the migrated area as compared with that of the control (FIG. 4).

Taken together, results of this invention indicate that GMAS is a potential lead compound for developing medicaments for treating cancers, particularly, the metastatic cancers, and the drug-resistant cancers.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of treating cancer in a subject comprising administering to the subject an effective amount of ganodermic acid S (GMAS) or a pharmaceutically acceptable salt thereof, wherein the cancer is any of colon cancer, hepatic cancer, breast cancer, or lung cancer.

2. The method of claim 1, wherein the lung cancer is resistant to gefitinib.

3. The method of claim 1, wherein the colon cancer is metastatic.

4. The method of claim 1, wherein the lung cancer is metastatic

5. The method of claim 1, further comprising subjecting the subject to radiation treatment after administering the ganodermic acid S or a pharmaceutically acceptable salt thereof to the subject.

6. The method of claim 1, further comprising administering to the subject another agent selected from the group consisting of anti-cancer drug, angiogenesis inhibitor, anti-virus agent, analgesic, anti-anemia drug, cytokine, granulocyte colony-stimulating factor (G-CSF), and anti-nausea drug.

7. The method of claim 1, wherein the subject is a human.
